(19) United States
(12) Patent Application Publication
(10) Pub. No.: US 2024/0277522 A1
(43) Pub. Date: Aug. 22, 2024

(54) SHUNTING SYSTEMS HAVING SHAPEABLE ELEMENTS AND ASSOCIATED DEVICES AND METHODS

(71) Applicant: Shifamed Holdings, LLC, Campbell, CA (US)

(72) Inventors: Eric Schultz, Los Altos, CA (US); Robert Chang, Belmont, CA (US); Katherine Sapozhnikov, Campbell, CA (US); Michelle Tran, Sunnyvale, CA (US); Tom Saul, Portland, OR (US); Tessa Bronez, Campbell, CA (US)

(21) Appl. No.: 18/291,656

(22) PCT Filed: Jul. 20, 2022
(86) PCT No.: PCT/US2022/037747

§ 371 Date: Jan. 24, 2024

Related U.S. Application Data
(60) Provisional application No. 63/227,869, filed on Jul. 30, 2021.
(60) Provisional application No. 63/292,164, filed on Dec. 21, 2021.

(51) Int. Cl. A61F 9/007

(52) U.S. Cl. CPC A61F 9/00781; A61F 2210/0004; A61F 2210/0019; A61F 2220/0008

(57) ABSTRACT

The present technology is generally directed to shunting systems, including shunting systems having a shapeable/conformable elongated housing or shunting element that can be contoured or otherwise selectively shaped to improve a fit with patient anatomy. The shunting systems and elongated housings described herein can have numerous other advantageous features expected to improve the operation of shunting systems, such as beveled leading edges, lateral outlets, suture rings, positioning appendages, and the like.

### CROSS-REFERENCE TO RELATED APPLICATION(S)

The present application claims priority to U.S. Provisional Patent Application No. 63/227,869, filed Jul. 30, 2021, and U.S. Provisional Patent Application No. 63/292,164, filed Dec. 21, 2021, the disclosures of which are incorporated by reference herein in their entireties.

### TECHNICAL FIELD

The present technology generally relates to implantable medical devices and, in particular, to shunting systems and associated methods for controlling fluid flow between a first body region and a second body region of a patient.

### BACKGROUND

Implantable shunting systems are widely used to treat a variety of patient conditions by shunting fluid from a first body region/cavity to a second body region/cavity. For example, shunting systems have been proposed for treating glaucoma. The flow of fluid through the shunting systems is primarily controlled by the pressure gradient across the shunt and the physical characteristics of the flow path defined through the shunt (e.g., the resistance of the shunt lumen). Conventional, early shunting systems (sometimes referred to as minimally invasive glaucoma shunts or “MIGS”) have shown clinical benefit; however, there is a need for improved shunting systems and techniques for addressing elevated intraocular pressure and risks associated with glaucoma. For example, there is a need for shunting systems capable of adjusting the therapy provided, including the flow rate between the two fluidly-connected bodies. As another example, there is a need for a shunting system capable of being modified after manufacture (e.g., in the clinic) to personalize the system for the patient and/or as part of the clinician's plan for the implant procedure.

### BRIEF DESCRIPTION OF THE DRAWINGS

Many aspects of the present technology can be better understood with reference to the following drawings. The components in the drawings are not necessarily drawn to scale. Instead, emphasis is placed on illustrating clearly the principles of the present technology. Furthermore, components can be shown as transparent in certain views for clarity of illustration only and not to indicate that the component is necessarily transparent. Components may also be shown schematically.

FIG. 1A illustrates a shunting system configured in accordance with select embodiments of the present technology.

FIG. 1B illustrates an elongated housing of the shunting system shown in FIG. 1A and configured in accordance with select embodiments of the present technology.

FIG. 1C illustrates a cross-section of the elongated housing shown in FIG. 1B and configured in accordance with select embodiments of the present technology.

FIG. 2 illustrates an elongated shapeable element of the shunting system shown in FIG. 1A and configured in accordance with select embodiments of the present technology.

FIGS. 3A and 3B illustrate another shunting system configured in accordance with select embodiment of the present technology.

FIGS. 3C and 3D illustrate the shunting system of FIGS. 3A and 3B implanted in a patient's eye in accordance with select embodiments of the present technology.

FIG. 4 illustrates another shunting system configured in accordance with select embodiments of the present technology.

FIG. 5A illustrates another shunting system configured in accordance with select embodiments of the present technology.

FIG. 5B illustrates still another shunting system configured in accordance with select embodiments of the present technology.

### DETAILED DESCRIPTION

The present technology is generally directed to shunting systems, including shunting systems having a shapeable/conformable elongated housing or shunting element that can be contoured or otherwise shaped to improve a fit with patient anatomy. For example, in some embodiments the shunting systems described herein include a shapeable/conformable element such as a spine element configured to at least partially control a shape of the elongated housing and/or shunting system. The shapeable element can be configured such that, if the shapeable element is deformed to change the shape of the elongated housing, the shapeable element retains the elongated housing in the changed shape for a selected period of time or indefinitely. Thus, the systems described herein can be shaped or otherwise manipulated into a desired position during an implant procedure to improve deliverability of the shunting systems into the patient, and to better match or fit patient anatomy once implanted. In some embodiments, the shunting systems described herein can be pre-shaped to match patient anatomy, in addition to or in lieu of having a shapeable/conformable element. The shunting systems and elongated housings described herein can have numerous other advantageous features expected to improve the delivery process and operation of shunting systems, such as beveled leading edges, lateral outlets, suture rings, positioning appendages, and the like, each of which are described in detail below.

In some embodiments, the shunting systems described herein can also selectively control fluid flow through the system to provide a titratable shunting therapy. For example, the shunting systems may include a plate assembly having one or more actuators for selectively controlling the flow of fluid through the shunting system and/or elongated housing. The actuators can be actuated after implanting the system to change the flow rate of fluid through the system.

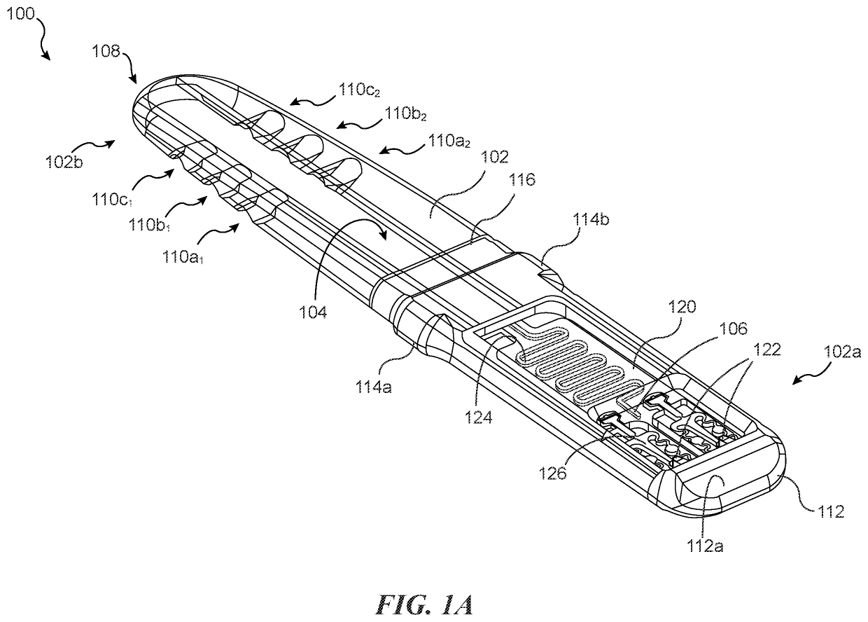

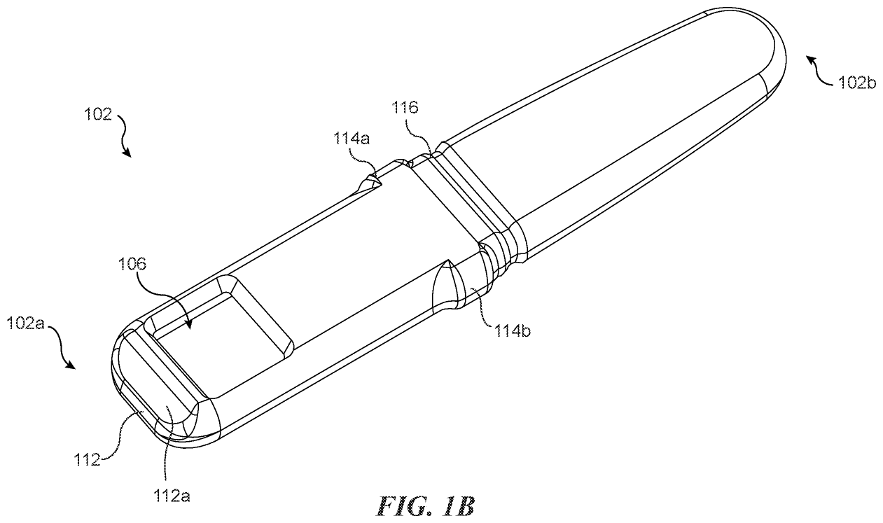

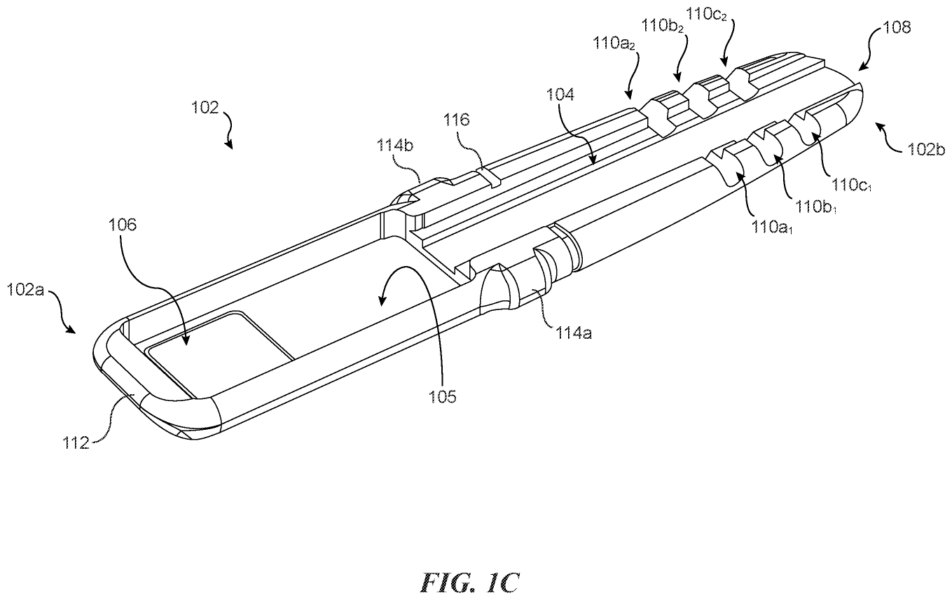

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the examples and claims but are not described in detail with respect to FIGS. 1A-5B.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

Reference throughout this specification to relative terms such as, for example, “generally,” “approximately,” and “about” are used herein to mean the stated value plus or minus 10%. Reference throughout this specification to the term “resistance” refers to fluid resistance unless the context clearly dictates otherwise. The terms “drainage rate” and “flow rate” are used interchangeably to describe the movement of fluid through a structure at a particular volumetric rate. The term “flow” is used herein to refer to the motion of fluid, in general.

Although certain embodiments herein are described in terms of shunting fluid from an anterior chamber of an eye, one of skill in the art will appreciate that the present technology can be readily adapted to shunt fluid from and/or between other portions of the eye, or, more generally, from and/or between a first body region and a second body region. Moreover, while the certain embodiments herein are described in the context of glaucoma treatment, any of the embodiments herein, including those referred to as “glaucoma shunts” or “glaucoma devices” may nevertheless be used and/or modified to treat other diseases or conditions, including other diseases or conditions of the eye or other body regions. For example, the systems described herein can be used to treat diseases characterized by increased pressure and/or fluid build-up, including but not limited to heart failure (e.g., heart failure with preserved ejection fraction, heart failure with reduced ejection fraction, etc.), pulmonary failure, renal failure, hydrocephalus, and the like. Moreover, while generally described in terms of shunting aqueous, the systems described herein may be applied equally to shunting other fluid, such as blood or cerebrospinal fluid, between the first body region and the second body region.

FIGS. 1A-1C illustrate a shunting system 100 (“the system 100”) configured in accordance with select embodiments of the present technology. More specifically, FIG. 1A is a perspective view of the system 100, FIG. 1B is a perspective view of an elongated housing 102 of the system 100, and FIG. 1C is a perspective cross-sectional view of the elongated housing 102 rotated 180 degrees about its longitudinal axis relative to FIG. 1B. As described in greater detail below, the system 100 is configured to provide a titratable therapy for draining fluid from a first body region, such as to drain aqueous from an anterior chamber of a patient's eye.

Referring collectively to FIGS. 1A-1C, the system 100 includes the elongated housing 102 and a flow control plate assembly 120. The elongated housing 102 (which can also be referred to as a casing, membrane, shunting element, or the like) extends between a first (e.g., proximal) end portion 102a and a second (e.g., distal) end portion 102b. The first end portion 102a of the elongated housing 102 includes a chamber 105 (FIG. 1C) and an opening 106. As shown in FIG. 1A, the flow control plate assembly 120 (which can also be referred to as a flow control plate, a flow control cartridge, a plate structure, plate assembly, or the like) is positioned within the chamber 105 of the elongated housing 102 and is configured to control the flow of fluid through the system 100. In particular, one or more fluid inlets or apertures 122 of the flow control plate assembly 120 align with the opening 106 in the elongated housing 102. The fluid inlets 122 permit fluid to enter an interior of the plate assembly 120 (and thus an interior of the elongated housing 102) from an environment external to the system 100. The flow control plate assembly 120 further includes one or more channels 124 for transporting fluid entering the plate assembly 120 via the fluid inlets 122 to a primary drainage lumen 104 of the elongated housing 102. In some embodiments, an upper surface of the plate assembly 120 forms a substantial fluid seal with an interior surface of the elongated housing 102 at the first end portion 102a such that the only way for fluid to enter the system 100 is through the fluid inlets 122. Accordingly, for fluid to flow through the system 100, it generally must flow through the plate assembly 120. The flow control plate assembly 120 can also include one or more actuators 126 (e.g., shape-memory actuators) for selectively controlling the flow of fluid through the plate assembly 120 (e.g., by selectively interfering with and not interfering with fluid flow through the inlets 122). In some embodiments, the flow control plate assembly 120, the actuators 126, and other associated elements can be generally similar to those described in U.S. Pat. Nos. 11,291,585, 11,166,849, and International Patent Application Nos. PCT/US22/13336, PCT/US20/55144, PCT/US20/55141, PCT/US21/14774, PCT/US21/18601, PCT/US21/23238, and PCT/US21/27742, the disclosures of which are incorporated by reference herein in their entireties and for all purposes.

Fluid flowing through the plate assembly 120 flows into the primary drainage lumen 104 as it moves toward the second end portion 102b of the system 100. The second end portion 102b can therefore include a number of outlets for draining fluid from the primary drainage lumen 104 to a desired drainage location (e.g., a bleb space). For example, the second end portion 102b can have an axial outlet or aperture 108 aligned with a longitudinal axis of the primary drainage lumen 104. The second end portion 102b can also have one or more lateral outlets or apertures 110a₁-110c₂ (collectively referred to herein as “the lateral outlets 110”) positioned along a side portion of the elongated housing 102 (the lateral outlets 110 are not shown in FIG. 1B). For example, in the illustrated embodiment, the elongated housing 102 includes a first pair of lateral outlets 110a₁ and 110a₂, a second pair of lateral outlets 110b₁ and 110b₂, and a third pair of lateral outlets 110c₁ and 110c₂. In some embodiments, the elongated housing 102 can have more or fewer pairs of lateral outlets, such as one, two, four, five, six, seven, eight, or more pairs. Moreover, in some embodiments, the elongated housing 102 only has lateral outlets 110 on one side, has lateral outlets 110 on both sides but laterally offset, and/or has an uneven number of lateral outlets 110 on each side.

In addition to facilitating draining of fluid, the lateral outlets 110 can also provide a visual cue to a physician implanting the device. For example, in some implant procedures and depending on patient anatomy, a physician may need to cut off some of the second end portion 102b of the elongated housing to make the system 100 “fit” in the patient. This may be done before or during the implant procedure. The lateral outlets 110 can have the same or substantially the same width (e.g., 0.1 mm, 0.2 mm, 0.3 mm, 0.4 mm, 0.5 mm, 0.6 mm, 0.7 mm, 0.8 mm, 0.9 mm, 1 mm, 2 mm, 3 mm, 4 mm, 5 mm, etc.) and can be spaced apart by a predetermined and known dimension (e.g., 0.1 mm, 0.2 mm, 0.3 mm, 0.4 mm, 0.5 mm, 0.6 mm, 0.7 mm, 0.8 mm, 0.9 mm, 1 mm, 2 mm, 3 mm, 4 mm, 5 mm, etc.). Accordingly, a physician can quickly determine the length of the elongated housing 102 that they will remove by counting the number of lateral outlets 110 that will be removed by their cut. For example, if the lateral outlets 110 have a length of 1 mm and are separated by 1 mm, each lateral outlet that is removed represents a 2 mm reduction in length of the system 100, in addition to the length of the elongated housing between the axial outlet 108 and the lateral outlet 110 closest to the axial outlet 108 (shown in FIGS. 1A and 1C as the third pair of lateral outlets 110c₁, 110c₂).

In some embodiments, the lateral outlets 110 also provide a natural hinge-point at which the elongated housing 102 can bend or otherwise flex. Without being bound by theory, enabling bending of the elongated housing 102 at the lateral outlets 110 is expected to allow the elongated housing 102 to better fit the anatomy of the patient (e.g., the curvature of the patient's eye). Indeed, in some embodiments the lateral outlets 110 need not be outlets, but instead can be thinned portions in the surface defining the elongated housing 102 that permit the elongated housing 102 to bend thereat.

The elongated housing 102 can include a number of other features that assist in delivering and positioning the system 100 in a patient. For example, the elongated housing 102 can include a beveled or tapered edge 112 at the first end portion 102a. Although the beveled edge 112 is shown as having an angled surface 112a extending from an upper surface of the system 100, in other embodiments the beveled edge 112 can take other suitable configurations in which the first end portion 102a converges toward a pointed/tapered end portion. For example, in some embodiments the beveled edge 112 can include an angled surface (not shown) extending from a lower surface of the system 100 in addition to or in lieu of the angled surface 112a extending from the upper surface of the system 100. During delivery, the beveled edge 112 (which is the leading edge during delivery of the system 100) helps the system 100 enter and pass through a slit or cut in patient tissue. For example, in the context of delivering the system 100 to the patient's eye to treat glaucoma, the beveled edge 112 can be inserted into a slit in the patient's sclera to assist with advancing the system 100 toward the patient's anterior chamber. Without being bound by theory, the beveled edge 112 is expected to reduce the complexity of delivering the system 100 into patient tissue, relative to other shunting systems with flat or non-beveled leading edges.

The elongated housing 102 further includes a first appendage, bumper, or stopper 114a and a second appendage, bumper, or stopper 114b (collectively referred to herein as “the appendages 114”). The appendages 114 can be positioned generally between the first end portion 102a and the second end portion 102b of the device, and can protrude laterally relative to a longitudinal axis of the elongated housing 102. The appendages 114 can also take the form of raised ring of material around the housing (not shown) or other surface modifications. In use, the appendages 114 can abut patient tissue to prevent the system 100 from advancing/migrating too far into the patient (e.g., past a target location within the patient), can be used to provide tactile feedback to a physician deploying the system 100, and/or may reduce and/or eliminate peritubular leakage around the system 100. For example, in the context of delivering the system 100 to the patient's eye to treat glaucoma, the appendages 114 can be configured to abut the edge of the patient's anterior chamber as the first end portion 102a of the system 100 is being advanced into the anterior chamber. The appendages 114 can therefore provide tactile feedback to the physician when the first end portion 102a is correctly positioned within the anterior chamber and/or prevent or at least reduce the first end portion 102a from being advanced too far into the anterior chamber. The appendages 114 can also reduce aqueous from leaking out of the anterior chamber around the system 100 (e.g., “peritubular leakage”) via the slit used to insert the first end portion 102a into the anterior chamber.

The elongated housing 102 further includes a suture ring 116. The suture ring 116 is an indentation or groove extending at least partially around the circumference of the elongated housing 102, and can be configured to receive a suture or other attachment mechanism for securing the system 100 to patient tissue following delivery of the system 100. The suture ring 116 can be configured such that, when a suture is secured around the suture ring 116, the suture does not impede or block flow of fluid through the system 100. For example, in some embodiments the system 100 may include a semi-rigid or rigid wire, ridge, or other element (not shown) extending around a perimeter of the suture ring 116 that can be configured to resist and/or prevent collapse of the elongated housing 102 if a suture is cinched too tightly around the suture ring 116. Moreover, although illustrated as extending around the full circumference of the elongated housing 102, in some embodiments the suture ring 116 extends only partially around the elongated housing 102, such as only across an upper surface of the elongated housing 102. Without being bound by theory, use of the suture ring 116 may enable the system 100 to be secured to patient tissue using a single suture, which is in turn expected to reduce the complexity of, and thus the time it takes to, implant and secure the system 100 in a desired position. In some embodiments, however, the elongated housing 102 may include multiple (e.g., two, three, four, or more) suture rings for securing the system 100 to patient tissue. The elongated housing 102 may further include additional features for securing the system 100 to patient tissue in addition to or in lieu of the suture ring 116. For example, the appendages 114 may include on or more apertures (not shown) configured to receive a suture.

In some embodiments, the one or more suture rings 116 may provide additional advantages to the system 100. For example, in addition to facilitating the securement of the system 100 to patient tissue, the suture rings 116 may also form a hinge-point at which the elongated housing 102 is configured to bend. Without being bound by theory, enabling bending of the elongated housing 102 at the suture rings 116 is expected to allow the elongated housing 102 to better fit the anatomy of the patient (e.g., the curvature of the patient's eye).

The elongated housing 102 can be composed of a slightly elastic or flexible biocompatible material (e.g., silicone, polydimethylsiloxane (PDMS), polymethylmethacrylate (PMMA), etc.). In some embodiments, the elongated housing 102 is at least partially shapeable such that, if deformed, it is configured to at least partially retain its deformed shape. For example, a physician may wish to at least partially bend the elongated housing 102 during or after implanting the system 100 into the patient to better match patient anatomy (e.g., the curvature of the patient's eye). The elongated housing 102 can be configured to retain any bending induced by the physician such that, following implantation, the elongated housing 102 (and thus the system 100) retains its deformed configuration. For example, in some embodiments the elongated housing 102 may include a shapeable or conformable element (not shown in FIGS. 1A-1C) that can at least partially determine a shape of the elongated housing 102 and that can also be deformed or bent by a clinician to change a shape of the elongated housing 102 during and/or after the implant procedure. In another example, other mechanisms could be provided for manipulating the shape of the elongated housing including, but not limited to, ratchets, screws, and structures that change shape with application of heat or fluid.

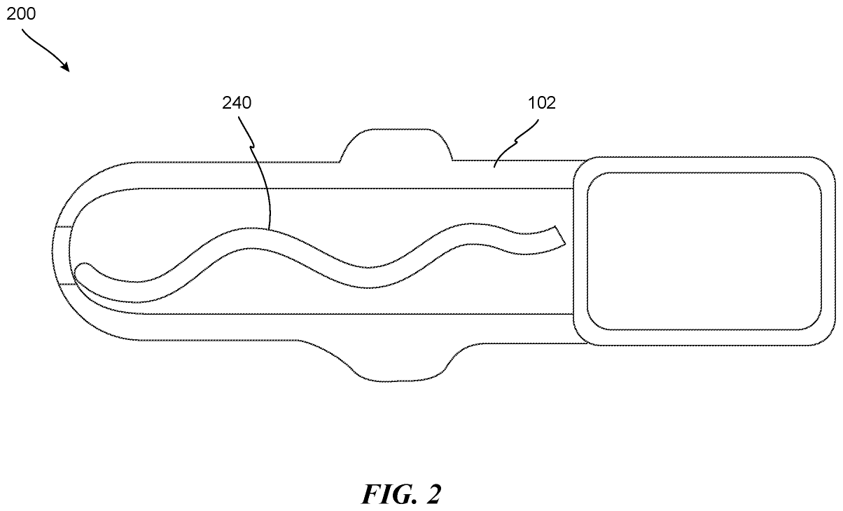

FIG. 2, for example, illustrates the elongated housing 102 having an elongated shapeable element 240 (“the shapeable element 240”). In some embodiments, the shapeable element 240 can be a malleable, pliable, or conformable band or spine that can be bent by a deformation force (e.g., a physician intentionally bending the system 100) into a different shape and, upon cessation of the deformation force, retain the different shape. In some embodiments, the shapeable element 240 can retain the selected shape indefinitely. In other embodiments, however, the shapeable element 240 may be composed of a material (e.g., hydrogel, polymer, etc.) with material properties that change over time such that the shapeable element 240 only retains the selected shape for a period of time (during delivery and for a selected period thereafter), and then assumes a different shape.

The shapeable element 240 can also be coupled to the elongated housing 102 such that, when the shapeable element 240 is deformed, the shape/orientation of the elongated housing 102 also changes. The shapeable element 240 can also be stiff enough such that it can at least partially resist deformation from relatively minor forces (e.g., to avoid unintentional deformation) and/or assist with delivering the device. The shapeable element 240 can be positioned anywhere in or along the elongated housing 102 for manipulation of the elongated housing's shape. It does not need to be along a “spine” or other specific location of the elongated housing 102. Moreover, although the shapeable element 240 is shown as having a serpentine shape, in other embodiments the shapeable element 240 can have other suitable shapes, such as linear, curved, or the like. Likewise, although shown as extending only partially along the length of the elongated housing 102, in other embodiments the shapeable element 240 can extend along substantially the full length of the elongated housing 102.

In some embodiments, the shapeable element 240 can be composed at least partially of polymers, hydrogel, gold, silver, titanium, platinum, rhodium, or other suitable materials. In some embodiment, the material(s) can be coated or plated with a second material, such as gold, platinum, rhodium, titanium, or polymers. The shapeability/conformability may also be achieved through composite structures that may include braiding, weaving, coiling, or the like. In other embodiments, the shapeable element 240 is omitted and the elongated housing 102 is itself composed of an at least partially shapeable material to assist in changing the shape of the elongated housing 102 for delivery and/or operation after implantation.

In operation, the system 100 may initially be delivered in a relatively straight or linear configuration (e.g., to fit within a delivery needle or other delivery apparatus). Once deployed into the patient, a physician can bend the system 100 to mold the system 100 to patient anatomy. For example, in the context of delivering the system 100 to the patient's eye to treat glaucoma, the physician can bend the system 100 to match the curvature of the patient's eye. The system 100 is configured to retain the bent shape based on the shapeable element 240. Without being bound by theory, this is expected to improve the “fit” of the system 100 in the patient.

In addition to facilitating preferential bending of the system 100 to better fit patient anatomy, the shapeable element 240 is also expected to increase the overall stiffness of the elongated housing 102 and/or the system 100. This is expected to simplify the delivery process by enabling a clinician to more readily push or otherwise deploy the system 100 out of a delivery apparatus (e.g., a needle, catheter, etc.) and/or push the system 100 into patient tissue (e.g., even when already deployed out of the delivery apparatus). Once the system 100 has been deployed from the delivery apparatus, the elongated housing 102 can be shaped/conformed to its desired configuration, and the shapeable element 240 is expected to help retain the elongated housing 102 at the desired configuration for a selected period of time as previously described. Thus, in some embodiments the shapeable element 240 is expected to both (a) provide a desirable stiffness to assist in delivering the device to a target implant location, and (b) provide a desirable shapeability/conformability to assist in retaining a desired configuration of the device to better match patient anatomy.

Although primarily described in terms of a metal or other solid structure, in some embodiments the shapeable element 240 can be composed of a stiffening element or material that undergoes a change in stiffness/compliance/conformability when implanted in the eye. For example, the stiffening material can be configured to transition from a first state before the system 100 is implanted in the eye to a second state when the system 100 is implanted in the eye. In some embodiments, the stiffening element transitions from the first state to and/or toward the second state in response to being exposed to patient fluids, such as aqueous or blood (e.g., via hydrating, liquifying, or the like) and/or body heat (e.g., via melting). In the first state, the stiffening material can cause the system 100 to have a first stiffness and a first compliance. In the second state, the stiffening material can cause the system 100 to have a second stiffness and a second compliance. The first stiffness is generally greater than the second stiffness and the first compliance is generally less than the second compliance. Accordingly, the stiffening element is configured to cause the stiffness of the system 100 to decrease and/or the compliance/conformability of the system 100 to increase after the device is implanted. This is expected to be beneficial because (a) the relatively higher stiffness/relatively lower compliance of the system 100 during implantation enables the system 100 to be deployed by a grasping tool or from a needle, catheter, or other delivery apparatus (e.g., by making it easier to “push” on the system 100 as compared with many conventional systems that are highly flexible/conformable before implantation and, accordingly, difficult to “push” through the needle or delivery apparatus), and (b) the relatively lower stiffness/relatively higher compliance of the system 100 after implantation enables the elongated body 102 of the system 100 to more easily conform to patient anatomy (e.g., due to forces imparted on the system 100 by patient anatomy) and/or enables a clinician to bend the elongated body 102 into a desired configuration following implantation.

The stiffening element can be any material configured to decrease the stiffness of the system 100 and/or increase the compliance of the system 100 following implantation of the system 100 into a patient's eye. For example, in some embodiments the stiffening element can be a material configured to transition from a solid (e.g., when in the first state) to a liquid (e.g., when in the second state). For example, the stiffening element can be a polysaccharide or other soluble element transitionable between a solid and a liquid state. In other embodiments, the stiffening element can be a solid in both the first state and the second state, but may nevertheless decrease in stiffness when implanted. For example, the stiffening element can be hydrogel or other material that loosens when exposed to liquid and/or heat. In some embodiments, the stiffening material remains within the elongated body 102 after it undergoes the change in stiffness. In other embodiments, the material can be flushed from the elongated body 102 after it undergoes the change in stiffness. In some embodiments, the stiffening element may by bio-resorbable such that it can be absorbed by the body when in the second state. Regardless of the material, the stiffening material may at least partially block one or more outflow ports (e.g., the axial outlet 108 and/or the lateral outlets 110, shown in FIGS. 1A and 1C) when in the first state. In such embodiments, the stiffening material may at least partially reduce the likelihood of hypotony occurring in the patient immediately following implantation of the system 100 (e.g., due to a partial time delay between being implanted and being cleared from the system 100).

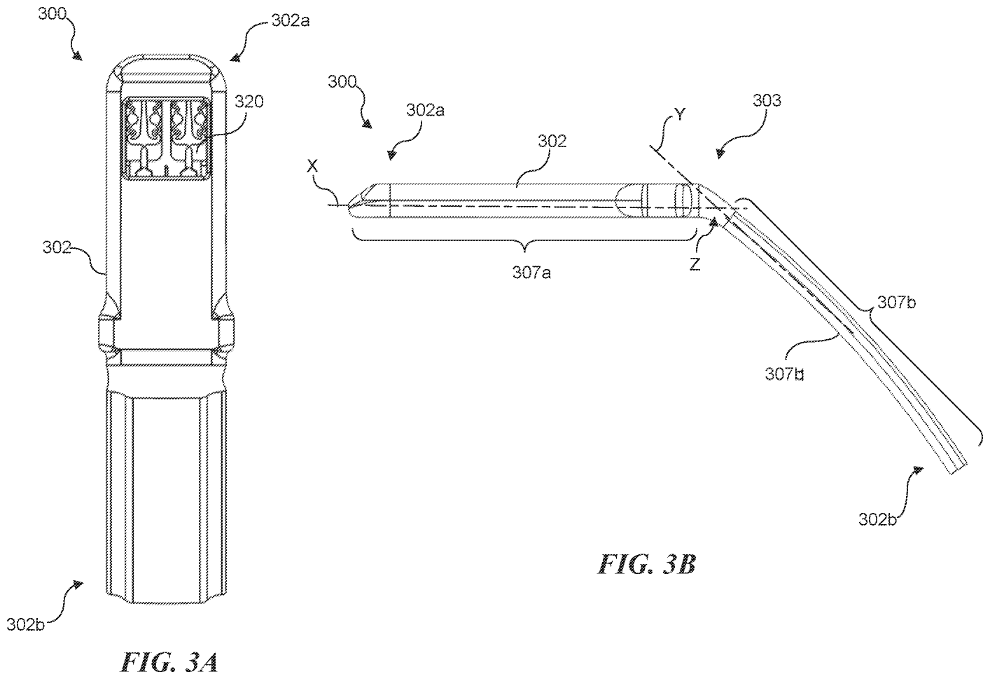

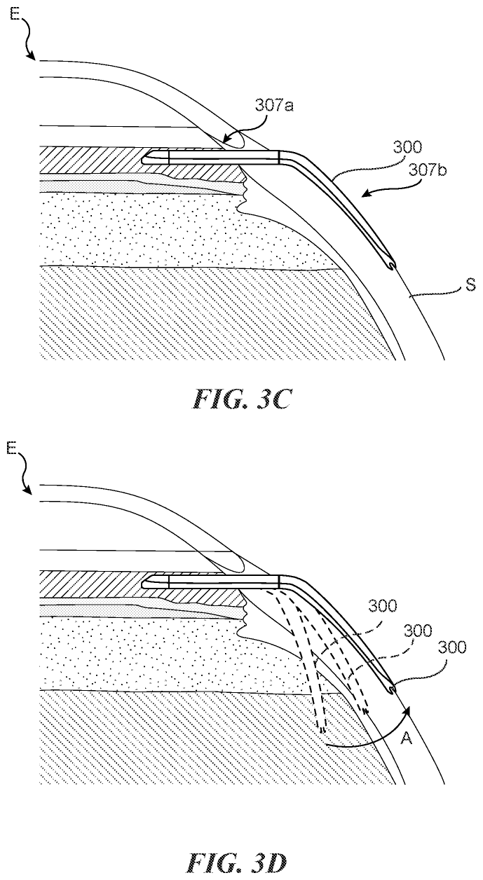

In some embodiments, the shunting systems described herein can be pre-shaped to fit patient anatomy, e.g., in addition to or in lieu of having a shapeable element. For example, FIGS. 3A-3D illustrate another shunting system 300 (“the system 300”) pre-shaped to fit patient anatomy and configured in accordance with select embodiments of the present technology. More specifically, FIG. 3A is a top view of the system 300, FIG. 3B is a side view of the system 300, FIG. 3C is a partial cross-sectional view of the system 100 implanted in an eye, and FIG. 3D is a partial cross-sectional view illustrating various stages of implanting the system 300 in the eye.

The system 300 can include certain features generally similar to those described above with respect to the system 100 (FIGS. 1A-2). For example, referring to FIG. 3A, the system 300 can include an elongated housing 302 extending between a first end region 302a and a second end region 302b. The first end region 302a of the elongated housing 302 can house a flow control plate assembly 320 that is configured to control the flow of fluid through the system 300. The flow control plate assembly 320 can be the same as or generally similar to the flow control plate assembly 120 shown in FIG. 1A. The system 300 can include other features described in detail above with reference to FIGS. 1A-1C.

Relative to the system 100, and as best shown in FIG. 3B, the system 300 is manufactured in a curved or bent configuration or shape. In the illustrated embodiment, the elongated housing 302 includes a hinge or bend region 303 positioned between the first end region 302a and the second end region 302b. The bend region 303 divides the elongated housing 302 into a first segment 307a extending between the first end region 302a and the bend region 303 and a second segment 307b extending between the second end region 302b and the bend region 303. Of note, a first axis X extending through the first segment 307a is angled relative to a second axis Y extending through the second segment 307b (as noted below, the second segment 307b can be curved, and so in some embodiments the second axis Y is defined as a best-fit line and/or an axis defined by only a portion of the second segment 307b adjacent the bend region 303, as shown in FIG. 3B). In some embodiments, an angle Z defined between the first segment 307a and the second segment 307b can be between about 90 degrees and 170 degrees, or between about 100 degrees and about 160 degrees, or between about 110 degrees and 150 degrees. In some embodiments, the angle Z is selected such that, when the first segment 307a resides within an interior of a patient's eye, the second segment 307b is angled relative to the first segment 307a to a degree that permits the second segment 307b to generally trace an outer surface of the patient's eye. As described in greater detail below with respect to FIG. 3D, in some embodiments the angle Z is selected such that, before the system 300 is implanted, the angle Z is less than the angle the system 300 will assume once implanted (e.g., the system 300 is “over curved” before being implanted and slightly straightens out during the implant procedure). As described below, this is expected to increase the anchoring force of the system 300 to the eye and keep the second segment 307b closely aligned with the sclera, thereby reducing and/or avoiding erosion or damage to the tendon and conjunctival tissue.

In addition to the bend region 303, the second segment 307b can also have a generally curved or arcuate shape configured to conform to patient anatomy, such as to match a curvature of an outer surface of an eye. For example, a surface 307b1 of the second segment 307b may have a slightly concave shape that conforms to the curvature of an eye. In some embodiments, both the first segment 307a and the second segment 307b have a curved or arcuate shape. In other embodiments, however, neither the first segment 307a nor the second segment 307b have a curved or arcuate shape. In some embodiments, the second segment 307b has a curve that is higher/greater than the curvature of the outer surface of the eye, such that when implanted, the second segment 307b at least partially “straightens out” to conform to the curvature of the outer surface of the eye. In embodiments in which the shunting element 310 is at least partially elastic, this is expected to increase the anchoring force between the system 300 and the eye and keep the second segment 307b closely aligned with the sclera, thereby reducing and/or avoiding erosion or damage to the tendon and conjunctival tissue.

Accordingly, as described above, the system 300 can be pre-curved or bent in two aspects: (1) the system 300 can include a hinge or bend region (e.g., bend region 303) that enables a first portion of the system 300 (e.g., the first segment 307a) to extend into a patient's eye and a second portion of the system 300 (e.g., the second segment 307b) to extend at an angle approximating an angle of an outer surface of the patient's eye, and (2) the first and or second portion of the system 300 can be curved to match the contour of the patient anatomy it is configured to be in apposition with (e.g., the curvature of the outer surface of the patient's eye). Without being bound by theory, having a pre-shaped system such as the system 300 is expected to reduce the need for a user to reshape or modify the system 300 during or after the implant procedure, e.g., by closely matching the shape of the system 300 to fit patient anatomy, such as the curvature of the patient's eye.

Despite being pre-shaped to fit patient anatomy, in some embodiments the elongated housing 302 can be composed of a semi-flexible or compliant material (e.g., silicone, PDMS, PMMA) that permits the elongated housing 302 to further assume an appropriate shape upon implantation. In some embodiments, the system 300 can also include a shapeable element (e.g., the shapeable element 240 described above with respect to FIG. 2) to further assist with fitting the system 300 to the patient's eye and/or to assist with deployment of the system 300 from a delivery instrument.

FIG. 3C illustrates the system 300 implanted in the patient's eye E. As illustrated, the first segment 307a is positioned within an interior of the patient's eye E while the second segment 307b extends along an outer surface S of the patient's eye E. As described with respect to FIG. 3B, the angle formed at the bend region 303 between the first segment 307a and the second segment 307b can be selected such that the second segment 307b lies substantially flat with the outer surface S of the patient's eye E. As also described with respect to FIG. 3B, the second segment 307b can be curved to match the curvature of the outer surface S of the patient's eye E. Although described as extending along an outer surface S of the patient's eye E, one skilled in the art will appreciate that the second segment 307b can be positioned within the eye while still tracing the curvature of the patient's eye, such as by being positioned between the sclera and the subconjunctiva. Accordingly, in some embodiments the outer surface S is an outer surface of the patient's sclera or other anatomical structure, and not an outer surface of the eye that is directly exposed to the external environment.

In some embodiments, the predefined angle between the first segment 307a and the second segment 307b can be less than the angle between the first segment 307a and the second segment 307b when the system 300 is implanted. For example, FIG. 3D illustrates various stages of an operation of implanting the system 300 into the patient's eye E in which the manufactured angle between the first segment 307a and the second segment 307b is less than the angle between the first segment 307a and the second segment 307b once the system 300 is implanted. In such embodiments, the system 300 can be flexed (e.g., in the direction indicted by arrow A) from its manufactured shape to its implanted shape during the implant procedure to conform to the curvature of the patient's eye. In such embodiments, the system 300 can be at least partially elastic such that flexing the system 300 to fit the curvature of the patient's eye helps hold the system 300 in a desired position or configuration (e.g., the system 300 is biased toward, but stretched beyond, its manufactured configuration once deployed, which is expected to increase the anchoring force of the system 300 to the eye). The flexing process described with respect to FIG. 3D is an optional step that may not be performed in some embodiments.

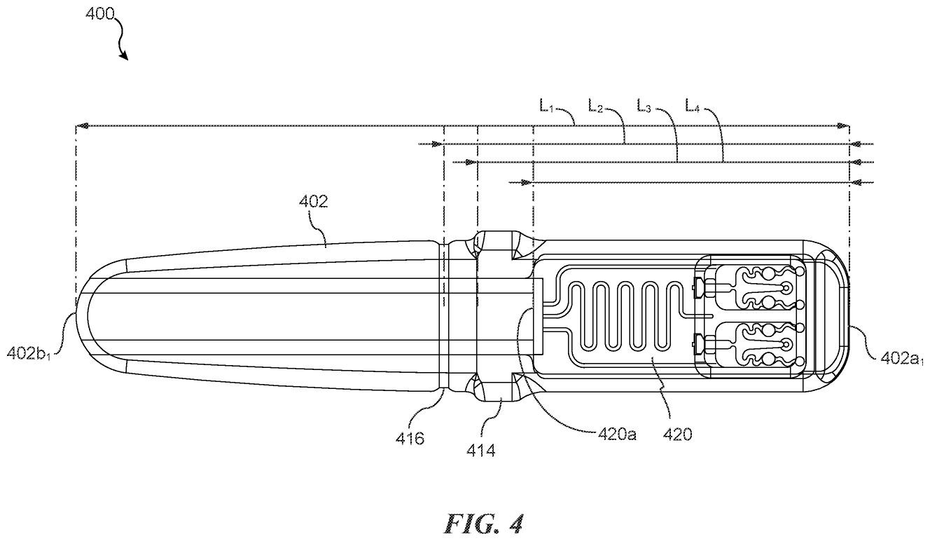

FIG. 4 is a top view of another shunting system 400 (“the system 400”) configured in accordance with select embodiments of the present technology. The system 400 includes features generally similar to the system 100 described above with reference to FIGS. 1A-1C. For example, the system 400 includes an elongated housing 402 and a flow control plate assembly 420. The elongated housing 402 and the flow control plate assembly 420 can be the same as, or substantially the same as, the elongated housing 102 and the flow control plate assembly 130 shown in FIGS. 1A-1C. For example, the elongated housing 402 can include one or more appendages 414 and a suture ring 416. Relative to the system 100, however, the system 400 does not include the lateral outlets 110.

The elongated housing 402 (and thus the system 400) can have an overall length L₁ between a first end 402a₁ and a second end 402b₁ of between about 4 mm and about 20 mm, such as between about 4 mm and 15 mm, or between about 4 mm and 12 mm, or between about 6 mm and 10 mm, or about 8 mm. A second length L₂ between the suture ring 416 and the first end 402a₁ can be between about 2 mm and about 8 mm, such as between about 2 mm and about 6 mm, or between about 3 mm and 6 mm, or between about 3 mm and 5 mm, or about 4 mm. A third length L₃ between the appendages 414 and the first end 402a₁ can also be between about 2 mm and about 8 mm, such as between about 2 mm and about 6 mm, or between about 3 mm and 6 mm, or between about 3 mm and 5 mm, or about 4 mm. A fourth length L₄ between a distal edge 420a of the plate assembly 420 and the first end 402a₁ can be between about 2 mm and about 8 mm, such as between about 2 mm and about 6 mm, or between about 3 mm and 6 mm, or between about 3 mm and 5 mm, or about 4 mm. The elongated housing 402 may also have a generally flat profile. For example, the elongated housing 402 may have a height that is less than about 2 mm, less than about 1 mm, less than about 0.5 mm, etc. The foregoing dimensions are provided merely as representative of certain embodiments, and other dimensions outside the ranges provided above are possible and included within the scope of the present technology. Indeed, the dimensions of the system 400 may be designed depending on the type of shunting system (e.g., glaucoma shunt vs. hydrocephalus shunt) and intended recipient (e.g., child vs. adult). As one skilled in the art will appreciate, any of the dimensions described with respect to the system 400 can also apply to the system 100 described with respect to FIGS. 1A-1C. Further, although the embodiments shown and described in conjunction with FIGS. 1A-4 are shunting systems for selectively adjusting a resistance to flow (and likewise flow rate), one of skill will appreciate that the present technology may be applied equally to other shunts and medical devices.

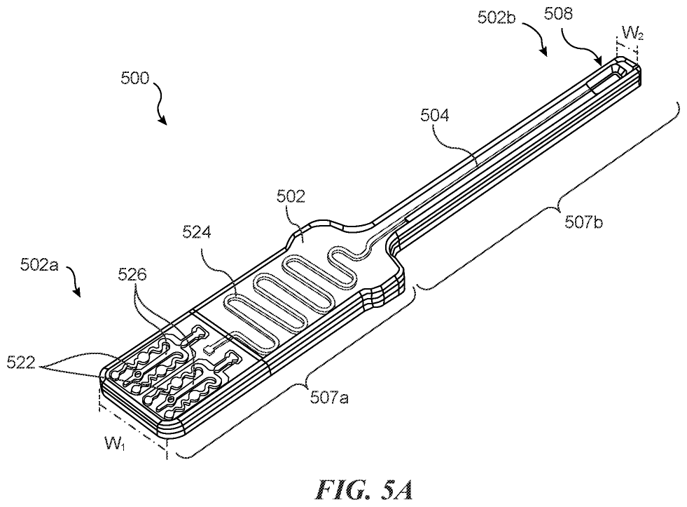

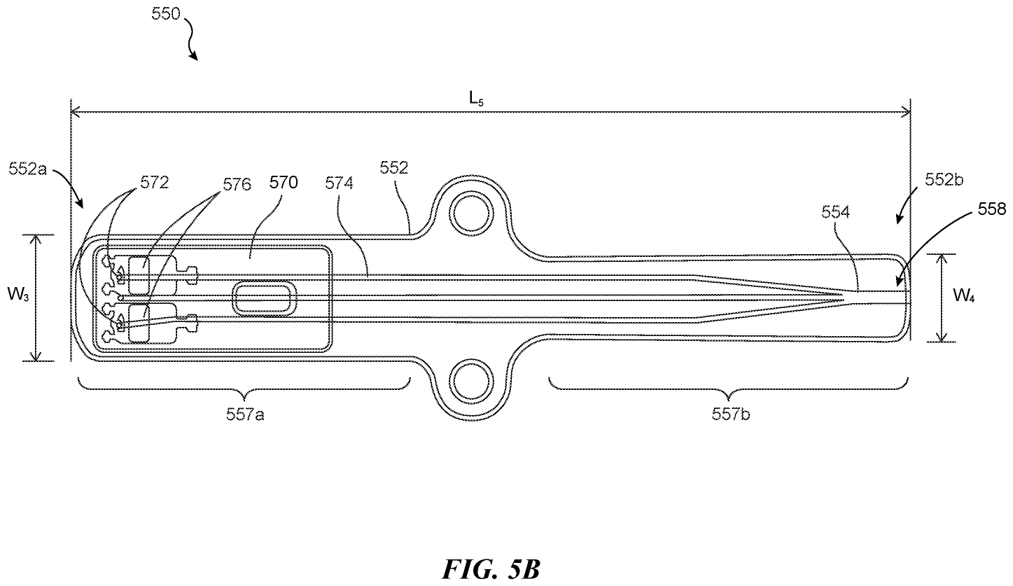

The shunting systems described herein can also have other suitable shapes and configurations. FIG. 5A, for example, is a perspective view of another shunting system 500 (“the system 500”) configured in accordance with select embodiments of the present technology. The system 500 includes certain features generally similar to the system 100 described above with reference to FIGS. 1A-1C. For example, the system 500 can include an elongated housing 502 defining one or more flow paths extending between a first end portion 502a and a second end portion 502b to shunt fluid therebetween. However, the system 500 includes a first segment 507a having a first width W₁, and a second segment 507b having a second width W₂ that is different than the first width W₁. In the illustrated embodiment, the second width W₂ is less than the first width W₁ (accordingly, the second segment 507b can also be referred to as a “narrow neck portion” or the like). The second width W₂ can be ¾, ½, ⅓, ¼, ⅛, or 1/16 of the first width W₁. In some embodiments, the first width W₁ can be between about 0.5 mm and about 2.5 mm, and the second width W₂ can be between about 0.5 mm and about 2 mm. Of course, the foregoing dimensions are provided merely as representative of certain embodiments, and other dimensions outside the ranges and values provided above are possible and included within the scope of the present technology. In some embodiments, incorporating a second segment having a smaller width than the other portions of the system 500 is expected to reduce the volume of space the system 500 occupies in the eye outside of the anterior cavity (e.g., relative to systems having an elongated housing with substantially constant width along its length), which in turn may reduce certain potential side effects of implanting the system 500 into the patient. Without being bound by theory, it is also expected that the reduced cross-sectional area of the second segment is expected to reduce its bending stiffness, thereby making it more conformable to the patient's eye. The reduced cross-sectional area of the second segment is also expected to provide improved anchoring relative to many conventional devices, which can inhibit/mitigate drift or migration of the system after implantation within the patient. In some embodiments, the second segment 507b can be angled or otherwise bent relative to the first segment 507a, as described with respect to the system 300 shown in FIGS. 3A-3D.

Despite having the narrow neck portion 508b, the system 500 can otherwise be generally similar to the system 100 described above. For example, the first end portion 502a of the elongated housing 502 can house or otherwise include certain features similar to or the same as the first end portion 102a of the system 100. For example, the first end portion 502a can include one or more fluid inlets 522 and one or more channels 524 for receiving fluid via the one or more inlets 522 (only one channel 524 is shown in FIG. 5A, but the system 500 can optionally include two, three, or more channels 524 as described with respect to the system 100 and as shown below with reference to FIG. 5B). The first end portion 502a of the system 500 can further house a flow control plate assembly 520 having one or more actuators 526 for selectively controlling the flow of fluid through the one or more fluid inlets 522. The second end portion 502b of the elongated housing 502 can include a primary drainage lumen 504, which can be fluidly coupled to and extend between the one or more channels 524 and an outflow aperture 508.

FIG. 5B is a top view of another shunting system 550 (“the system 550”) configured in accordance with select embodiments of the present technology. The system 550 includes a number of features generally similar to the system 500 described above with reference to FIG. 5A and the system 100 described above with reference to FIGS. 1A-1C. For example, the system 550 includes an elongated housing 552 defining one or more flow paths extending between a first end portion 552a and a second end portion 552b to shunt fluid therebetween. The first end portion 552a can include one or more fluid inlets 572 and one or more channels 574 for receiving fluid via the one or more inlets 572 (three channels 574 are shown in FIG. 5B, but the system 550 can optionally include one, two, four, or more channels 574 as described previously). One aspect of the system 550 that differs from the system 500 described above with reference to FIG. 5A is that the channels 574 are generally straight/linear extending along the elongated housing 552 between the first end portion 552a and the second end portion 552b (rather than having a serpentine arrangement like channel 524 of the system 500 of FIG. 5A).

The first end portion 552a of the system 550 can further house a flow control plate assembly 570 having one or more actuators 576 for selectively controlling the flow of fluid through the one or more fluid inlets 572. The second end portion 552b of the elongated housing 552 can include a primary drainage lumen 554, which can be fluidly coupled to and extend between the one or more channels 574 and an outflow aperture 558.

The elongated housing 552 (and thus the system 550) can have an overall length L₅ of between about 8 mm and 12 mm, such as between about 9 mm and 11 mm, or between about 10 mm and 11 mm, or between about 10.5 mm and 11 mm. In one particular embodiment, the length L₅ is 10.8 mm. The system 550 includes a first segment 557a having a first width W₃ and a length of between about 4 mm and 5 mm (e.g., 4.36 mm), and a second segment 557b having a second width W₄ less than the first width W₃ and a length of between about 4 mm and 5 mm (e.g., 4.77 mm). In some embodiments, the first width W₃ can be between about 1 mm and about 2 mm (e.g., 1.63 mm), and the second width W₄ can be between about 0.5 mm and about 1.5 mm (e.g., 1.13 mm). Similar to the devices described previously, the elongated housing 552 may also have a generally flat profile. For example, the elongated housing 552 may have a height that is less than about 2 mm, less than about 1 mm, less than about 0.5 mm, etc. The foregoing dimensions are provided merely as representative of certain embodiments, and other dimensions outside the ranges provided above are possible and included within the scope of the present technology. Indeed, the dimensions of the system 550 may be designed depending on the type of shunting system (e.g., glaucoma shunt vs. hydrocephalus shunt) and intended recipient (e.g., child vs. adult). As one skilled in the art will appreciate, any of the dimensions described with respect to the system 550 can also apply to the system 100 described with respect to FIGS. 1A-1C and the system 500 described with respect to FIG. 5A. Further, although the embodiments shown and described in conjunction with FIGS. 1A-5B are shunting systems for selectively adjusting a resistance to flow (and likewise flow rate), one of skill will appreciate that the present technology may be applied equally to other shunts and medical devices.

The systems described herein can be implanted at any suitable position or location within the eye that fluidly connects the anterior cavity to a desired outflow location, such as a subconjunctival bleb space. In some embodiments, for example, the systems described herein can be positioned such that an inflow region of the shunting system is within the anterior cavity and “above” (e.g., superficially positioned relative to) the iris in the anterior chamber. In such embodiments, any actuators carried by the system (e.g., the actuator 126 of the system 100, the actuator 526 of the system 500, the actuator 576 of the system 550) may be directly visible from the exterior of the eye, and can therefore be directly targeted using a suitable energy modality (e.g., laser energy). In other embodiments, the systems described herein can be positioned such that the inflow region of the shunting system is within the anterior cavity but “under” (e.g., posteriorly positioned relative to) the iris in the posterior chamber. Without being bound by theory, positioning the inflow region of the shunting system under the iris in the posterior chamber may reduce the likelihood of endothelial cell loss due to the implant. However, implanting the system such that the inflow region is under the iris may provide other challenges. For example, one or more inflow apertures (e.g., the fluid inlets 122 of the system 100, the fluid inlets 522 of the system 500, the fluid inlets 572 of the system 550) of the system may need to be repositioned such that they are on the side of or underneath the system to ensure that they remain in fluid communication with the anterior cavity. Moreover, positioning the inflow region of the shunting system under the iris may “block” the actuators and make them more challenging to locate and therefore actuate. In such embodiments, the actuators can nevertheless be identified and actuated by (a) performing an iridectomy (e.g., permanently cutting away a portion of the iris) to provide a pathway for laser or other forms of energy from an energy source positioned external to the eye to reach the actuators, (b) dilating the pupil before actuation to expose the actuators to energy from the energy source, and/or (c) utilizing a first energy modality that has a first wavelength transparent to the iris to locate the actuator (e.g., similar to using sonar to identify objects submerged in water), and then utilizing a second energy modality having a second wavelength to energize the actuator without substantially heating or otherwise affecting the iris. However, as one skilled in the art will appreciate from the disclosure herein, the systems described above are not limited to any particular location or position, and therefore the present technology is not intended to be limited by the foregoing description. Indeed, the shunting systems described herein can be positioned in other parts of the eye or, more generally, in or at other parts of the body for draining fluid from a first body region to a second body region.

### EXAMPLES

Several aspects of the present technology are set forth in the following examples:
1. An implantable shunting element for treating a patient, the implantable shunting element comprising:
   a first end portion and a second end portion spaced apart from the first end portion by a body of the shunting element;
   a drainage lumen extending at least partially between the first end portion and the second end portion for transporting fluid therebetween; and
   a conformable portion between the first end portion and the second end portion,
   wherein the conformable portion is configured to facilitate bending of the shunting element to a desired shape corresponding, at least in part, to an anatomical structure of the patient.
2. The implantable shunting element of example 1 wherein the conformable portion comprises one or more lateral outlets or apertures at the second end portion of the shunting element.
3. The implantable shunting element of example 1 wherein the conformable portion comprises one or more thinned portions in an exterior surface of the body of the second end portion of the shunting element.
4. The implantable shunting element of example 1 wherein the conformable portion comprises one or more suture rings extending at least partially around a circumference of the body of the second end portion of the shunting element.
5. The implantable shunting element of example 1 wherein the conformable portion comprises one or more indentations or grooves extending at least partially around a circumference of the body of the second end portion of the shunting element.
6. The implantable shunting element of any of examples 1-5 wherein:
   the first end portion of the shunting element has a first width, and the second end portion of the shunting element has a second width less than the first width; and
   the conformable portion comprises at least a region of the second end portion of the shunting element having the second width.
7. The implantable shunting element of any of examples 1-6 wherein the conformable portion is configured to facilitate bending of the shunting element to a desired shape before implantation of the shunting element within the patient.
8. The implantable shunting element of any of examples 1-7 wherein the conformable portion is configured to facilitate bending of the shunting element to correspond, at least in part, to curvature of the patient's eye.
9. A shunting element for treating a patient, the shunting element comprising:
   a first end portion;
   a second end portion spaced apart from the first end portion by a body of the shunting element;
   a drainage lumen extending at least partially between the first end portion and the second end portion for transporting fluid therebetween; and
   a soluble stiffening element carried by the shunting element, wherein the stiffening element is configured to (a) impart a first stiffness to the shunting element before and during implantation of the shunting element, and (b) impart a second stiffness less than the first stiffness to the shunting element after implantation of the shunting element.
10. The shunting element of example 9 wherein the stiffening element is configured to transition from a first state that imparts the first stiffness to and/or toward a second state that imparts the second stiffness when exposed to patient fluid.
11. The shunting element of example 9 or example 10 wherein the stiffening element is solid in the first state and liquid in the second state.
12. The shunting element of example 9 or example 10 wherein the stiffening element is solid in the first state and the second state.
13. The shunting element of example 9 wherein the stiffening element is configured to transition from a first state that imparts the first stiffness to and/or toward a second state that imparts the second stiffness when exposed to body heat.
14. The shunting element of any one of examples 9-13 wherein the stiffening element is composed of hydrogel.
15. The shunting element of any one of examples 9-13 wherein the stiffening element is composed of polysaccharide.
16. The shunting element of any one of examples 9-15 wherein the stiffening element is positioned within the shunting element.
17. The shunting element of any one of examples 9-16 wherein the stiffening element is composed of a bio-resorbable material.
18. A shunting element for treating a patient, comprising:
   a first end portion;
   a second end portion spaced apart from the first end portion by a body of the shunting element; and
   a bend region between the first end portion and the second end portion, wherein the bend region defines (a) a first segment of the shunting element extending between the first end portion and the bend region and (b) a second segment of the shunting element extending between the second end portion and the bend region,
   wherein a first longitudinal axis extending through the first segment is angled relative to a second longitudinal axis extending through the second segment.
19. The shunting element of example 18 wherein the first longitudinal axis and the second longitudinal axis form an angle between about 90 degrees and about 170 degrees.
20. The shunting element of example 19 wherein the shunting element is configured such that the angle increases when the shunting element is implanted in the patient.
21. The shunting element of example 19 wherein the shunting element is configured such that the angle remains substantially the same when the shunting element is implanted in the patient.
22. The shunting element of any one of examples 18-21 wherein the second segment includes a curved surface configured to mate with a corresponding curved surface of a patient's eye.
23. The shunting element of example 22 wherein the curved surface has a curvature configured to match a curvature of the corresponding curved surface of the patient's eye.
24. The shunting element of example 23 wherein the curved surface is configured to have a higher curvature than a curvature of the corresponding curved surface of the patient's eye, and wherein, when implanted, the curved surface is configured to assume a new curvature approximating the curvature of the corresponding curved surface of the patient's eye.
25. A shunting element for treating a patient, the shunting element comprising:
   a first end portion and a second end portion spaced apart from the first end portion by a body of the shunting element;
   a drainage lumen extending at least partially between the first end portion and the second end portion for transporting fluid therebetween; and
   a shapeable element carried by the shunting element and extending at least partially between the first end portion and the second end portion,
   wherein the shapeable element at least partially controls a shape of the shunting element, and wherein the shapeable element is configured such that deformation thereof changes the shape of shunting element and retains the shunting element in the changed shape for a predetermined period of time.
26. The shunting element of example 25 wherein the shapeable element is at least partially composed of a polymer and/or hydrogel.
27. The shunting element of example 25 or example 26 wherein the shapeable element is configured to transition from a first state that imparts a first stiffness to and/or toward a second state that imparts a second stiffness when exposed to patient fluid.
28. The shunting element of any one of examples 25-27 wherein the shapeable element is solid in the first state and liquid in the second state.
29. The shunting element of example 27 wherein the shapeable element is solid in the first state and the second state.
30. The shunting element of example 25 wherein the shapeable element is at least partially composed of gold and/or silver.
31. The shunting element of any one of examples 25-30 wherein the second end portion includes a beveled edge.
32. The shunting element of any one of examples 25-31, further comprising one or more grooves extending at least partially around an outer circumference of the shunting element, wherein the one or more grooves are configured to receive a suture to secure the shunting element to patient tissue.
33. The shunting element of example 32 wherein the one or more grooves extend around an entire circumference of the shunting element.
34. The shunting element of example 32 or example 33 wherein the shunting element is configured to hinge or otherwise bend at the one or more grooves.
35. The shunting element of any one of examples 25-34, further comprising one or more appendages extending laterally relative to a longitudinal axis of the shunting element, wherein the one or more appendages are configured to abut patient tissue when the shunting element is implanted.
36. The shunting element of example 35 wherein the one or more appendages are configured to reduce peritubular leakage when the shunting element is implanted in the patient.
37. The shunting element of any one of examples 25-36, further comprising a plurality of outlets fluidly coupled to the drainage lumen and positioned laterally along a side of the shunting element.
38. The shunting element of example 37 wherein the plurality of outlets includes a first plurality of outlets positioned along a first side of the shunting element and a second plurality of outlets positioned along a second side of the shunting element.
39. The shunting element of example 37 or example 38 wherein individual outlets of the plurality of outlets are spaced apart by a predetermined increment.
40. The shunting element of any one of examples 25-39 wherein the shunting element includes a chamber configured to house at least one shape memory actuator for selectively controlling flow of fluid through the shunting element.
41. The shunting element of any one of examples 25-40 wherein the chamber is configured to house a plate assembly having the at least one shape memory actuator.
42. A shunting system for treating a patient, the shunting system comprising:
   an elongated housing extending between a first end portion and a second end portion, the elongated housing including—
      a chamber, and
      a drainage lumen fluidly extending between the chamber and the second end portion;
   a shape memory actuator positioned within the chamber and configured to at least partially control a flow of fluid through the chamber; and
   a conformable element configured to at least partially control a shape of the elongated housing,
   wherein, if the conformable element is deformed to change the shape of the elongated housing, the conformable element is configured to retain the elongated housing in the changed shape for a selected period of time after the shunting system is implanted within the patient.
43. The shunting system of example 42 wherein the conformable element is at least partially composed of a hydrogel, a polymer, gold, and/or silver.
44. The shunting element of example 42 or example 43 wherein, when exposed to patient fluid, the conformable element is configured to transition from (a) a first state that imparts a first stiffness to and/or toward (b) a second state that imparts a second stiffness less than the first stiffness.
45. The shunting element of example 44 wherein the conformable element is solid in the first state and liquid in the second state.
46. The shunting element of example 44 wherein the conformable element is solid in the first state and solid in the second state.
47. The shunting system of any one of examples 42-46 wherein the elongated housing further includes one or more appendages extending laterally relative to a longitudinal axis of the elongated housing, and wherein the one or more appendages are configured to abut patient tissue when the shunting system is implanted within an eye of the patient.
48. The shunting system of example 47 wherein the one or more appendages are configured to reduce peritubular leakage when the system is implanted within an eye of the patient.
49. The shunting system of any one of examples 42-48, further comprising a plurality of outlets fluidly coupled to the drainage lumen and positioned laterally along a side of the elongated housing, wherein individual outlets of the plurality of outlets are spaced apart by a predetermined increment.
50. The shunting system of example 49 wherein the plurality of outlets includes a first plurality of outlets positioned along a first side of the elongated housing and a second plurality of outlets positioned along a second side of the elongated housing.
51. A method of implanting a shunting element in a patient, the method comprising:
   delivering the shunting element to a target location within the patient; and
   selectively adjusting a shape of the shunting element to conform to patient anatomy, wherein the shunting element includes a shapeable element that retains the shunting element in the adjusted shape for a preselected period of time.
52. The method of example 51 wherein the target location is within an eye of the patient, and wherein adjusting the shape of the shunting element includes bending the shunting element to conform to a curvature of the patient's eye.
53. The method of example 51 or example 52 wherein delivering the shunting element to a target location within the patient includes advancing the shunting element until one or more appendages of the shunting element abut patient tissue.
54. The method of any one of examples 51-53 wherein the shapeable element is composed of a soluble material, and wherein the shapeable element is configured to (a) impart a first stiffness to the shunting element during delivery of the shunting element to the target location within the patient and for a first preselected period of time after delivery, and (b) impart a second stiffness less than the first stiffness to the shunting element for a second preselected period of time after the first period of time.
55. The method of any one of examples 51-54 wherein the shapeable element is composed, at least in part, of hydrogel.
56. The method of any one of examples 51-54 wherein the shapeable element is composed, at least in part, of polysaccharide.

### Conclusion

The above detailed description of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, any of the features of the intraocular shunts described herein may be combined with any of the features of the other intraocular shunts described herein and vice versa. Moreover, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions associated with intraocular shunts have not been shown or described in detail to avoid unnecessarily obscuring the description of the embodiments of the technology. Where the context permits, singular or plural terms may also include the plural or singular term, respectively.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. An implantable shunting element for treating a patient, the implantable shunting element comprising:
a first end portion and a second end portion spaced apart from the first end portion by a body of the shunting element;
a drainage lumen extending at least partially between the first end portion and the second end portion for transporting fluid therebetween; and
a conformable portion between the first end portion and the second end portion,
wherein the conformable portion is configured to facilitate bending of the shunting element to a desired shape corresponding, at least in part, to an anatomical structure of the patient.

2. The implantable shunting element of claim 1 wherein the conformable portion comprises one or more lateral outlets or apertures at the second end portion of the shunting element.

3. The implantable shunting element of claim 1 wherein the conformable portion comprises one or more thinned portions in an exterior surface of the body of the second end portion of the shunting element.

4. The implantable shunting element of claim 1 wherein the conformable portion comprises one or more suture rings extending at least partially around a circumference of the body of the second end portion of the shunting element.

5. The implantable shunting element of claim 1 wherein the conformable portion comprises one or more indentations or grooves extending at least partially around a circumference of the body of the second end portion of the shunting element.

6. The implantable shunting element of claim 1 wherein:
the first end portion of the shunting element has a first width, and the second end portion of the shunting element has a second width less than the first width; and
the conformable portion comprises at least a region of the second end portion of the shunting element having the second width.

7. The implantable shunting element of claim 1 wherein the conformable portion is configured to facilitate bending of the shunting element to a desired shape before implantation of the shunting element within the patient.

8. The implantable shunting element of claim 1 wherein the conformable portion is configured to facilitate bending of the shunting element to correspond, at least in part, to curvature of the patient's eye.

9. A shunting element for treating a patient, the shunting element comprising:
a first end portion;
a second end portion spaced apart from the first end portion by a body of the shunting element;
a drainage lumen extending at least partially between the first end portion and the second end portion for transporting fluid therebetween; and
a soluble stiffening element carried by the shunting element, wherein the stiffening element is configured to (a) impart a first stiffness to the shunting element before and during implantation of the shunting element, and (b) impart a second stiffness less than the first stiffness to the shunting element after implantation of the shunting element.

10. The shunting element of claim 9 wherein the stiffening element is configured to transition from a first state that imparts the first stiffness to and/or toward a second state that imparts the second stiffness when exposed to patient fluid.

11. The shunting element of claim 10 wherein the stiffening element is solid in the first state and liquid in the second state.

12. The shunting element of claim 10 wherein the stiffening element is solid in the first state and the second state.

13. The shunting element of claim 9 wherein the stiffening element is configured to transition from a first state that imparts the first stiffness to and/or toward a second state that imparts the second stiffness when exposed to body heat.

14. The shunting element of claim 9 wherein the stiffening element is composed of hydrogel.

15. The shunting element of claim 9 wherein the stiffening element is composed of polysaccharide.

16. The shunting element of claim 9 wherein the stiffening element is positioned within the shunting element.

17. The shunting element of claim 9 wherein the stiffening element is composed of a bio-resorbable material.

18. A shunting element for treating a patient, comprising:
a first end portion;
a second end portion spaced apart from the first end portion by a body of the shunting element; and
a bend region between the first end portion and the second end portion, wherein the bend region defines (a) a first segment of the shunting element extending between the first end portion and the bend region and (b) a second segment of the shunting element extending between the second end portion and the bend region,
wherein a first longitudinal axis extending through the first segment is angled relative to a second longitudinal axis extending through the second segment.

19. The shunting element of claim 18 wherein the first longitudinal axis and the second longitudinal axis form an angle between about 90 degrees and about 170 degrees.

20. The shunting element of claim 19 wherein the shunting element is configured such that the angle increases when the shunting element is implanted in the patient.

21. The shunting element of claim 19 wherein the shunting element is configured such that the angle remains substantially the same when the shunting element is implanted in the patient.

22. The shunting element of claim 18 wherein the second segment includes a curved surface configured to mate with a corresponding curved surface of a patient's eye.

23. The shunting element of claim 22 wherein the curved surface has a curvature configured to match a curvature of the corresponding curved surface of the patient's eye.

24. The shunting element of claim 23 wherein the curved surface is configured to have a higher curvature than a curvature of the corresponding curved surface of the patient's eye, and wherein, when implanted, the curved surface is configured to assume a new curvature approximating the curvature of the corresponding curved surface of the patient's eye.

25. A shunting element for treating a patient, the shunting element comprising:
a first end portion and a second end portion spaced apart from the first end portion by a body of the shunting element;
a drainage lumen extending at least partially between the first end portion and the second end portion for transporting fluid therebetween; and
a shapeable element carried by the shunting element and extending at least partially between the first end portion and the second end portion,
wherein the shapeable element at least partially controls a shape of the shunting element, and wherein the shapeable element is configured such that deformation thereof changes the shape of shunting element and retains the shunting element in the changed shape for a predetermined period of time.

26. The shunting element of claim 25 wherein the shapeable element is at least partially composed of a polymer and/or hydrogel.

27. The shunting element of claim 26 wherein the shapeable element is configured to transition from a first state that imparts a first stiffness to and/or toward a second state that imparts a second stiffness when exposed to patient fluid.

28. The shunting element of claim 27 wherein the shapeable element is solid in the first state and liquid in the second state.

29. The shunting element of claim 26 wherein the shapeable element is solid in the first state and the second state.

30. The shunting element of claim 25 wherein the shapeable element is at least partially composed of gold and/or silver.

31. The shunting element of claim 25 wherein the second end portion includes a beveled edge.

32. The shunting element of claim 25, further comprising one or more grooves extending at least partially around an outer circumference of the shunting element, wherein the one or more grooves are configured to receive a suture to secure the shunting element to patient tissue.

33. The shunting element of claim 32 wherein the one or more grooves extend around an entire circumference of the shunting element.

34. The shunting element of claim 32 wherein the shunting element is configured to hinge or otherwise bend at the one or more grooves.

35. The shunting element of claim 25, further comprising one or more appendages extending laterally relative to a longitudinal axis of the shunting element, wherein the one or more appendages are configured to abut patient tissue when the shunting element is implanted.

36. The shunting element of claim 35 wherein the one or more appendages are configured to reduce peritubular leakage when the shunting element is implanted in the patient.

37. The shunting element of claim 25, further comprising a plurality of outlets fluidly coupled to the drainage lumen and positioned laterally along a side of the shunting element.

38. The shunting element of claim 37 wherein the plurality of outlets include a first plurality of outlets positioned along a first side of the shunting element and a second plurality of outlets positioned along a second side of the shunting element.

39. The shunting element of claim 37 wherein individual outlets of the plurality of outlets are spaced apart by a predetermined increment.

40. The shunting element of claim 25 wherein the shunting element includes a chamber configured to house at least one shape memory actuator for selectively controlling flow of fluid through the shunting element.

41. The shunting element of claim 40 wherein the chamber is configured to house a plate assembly having the at least one shape memory actuator.

42. A shunting system for treating a patient, the shunting system comprising:
an elongated housing extending between a first end portion and a second end portion, the elongated housing including—
chamber, and
a drainage lumen fluidly extending between the chamber and the second end portion;
a shape memory actuator positioned within the chamber and configured to at least partially control a flow of fluid through the chamber; and
a conformable element configured to at least partially control a shape of the elongated housing,
wherein, if the conformable element is deformed to change the shape of the elongated housing, the conformable element is configured to retain the elongated housing in the changed shape for a selected period of time after the shunting system is implanted within the patient.

43. The shunting system of claim 42 wherein the conformable element is at least partially composed of a hydrogel, a polymer, gold, and/or silver.

44. The shunting element of claim 42 wherein, when exposed to patient fluid, the conformable element is configured to transition from (a) a first state that imparts a first stiffness to and/or toward (b) a second state that imparts a second stiffness less than the first stiffness.

45. The shunting element of claim 44 wherein the conformable element is solid in the first state and liquid in the second state.

46. The shunting element of claim 44 wherein the conformable element is solid in the first state and solid in the second state.

47. The shunting system of claim 42 wherein the elongated housing further includes one or more appendages extending laterally relative to a longitudinal axis of the elongated housing, and wherein the one or more appendages are configured to abut patient tissue when the shunting system is implanted within an eye of the patient.

48. The shunting system of claim 47 wherein the one or more appendages are configured to reduce peritubular leakage when the system is implanted within an eye of the patient.

49. The shunting system of claim 42, further comprising a plurality of outlets fluidly coupled to the drainage lumen and positioned laterally along a side of the elongated housing, wherein individual outlets of the plurality of outlets are spaced apart by a predetermined increment.

50. The shunting system of claim 49 wherein the plurality of outlets include a first plurality of outlets positioned along a first side of the elongated housing and a second plurality of outlets positioned along a second side of the elongated housing.

51. A method of implanting a shunting element in a patient, the method comprising:
delivering the shunting element to a target location within the patient; and
selectively adjusting a shape of the shunting element to conform to patient anatomy, wherein the shunting element includes a shapeable element that retains the shunting element in the adjusted shape for a preselected period of time.

52. The method of claim 51 wherein the target location is within an eye of the patient, and wherein adjusting the shape of the shunting element includes bending the shunting element to conform to a curvature of the patient's eye.

53. The method of claim 51 wherein delivering the shunting element to a target location within the patient includes advancing the shunting element until one or more appendages of the shunting element abut patient tissue.

54. The method of claim 51 wherein the shapeable element is composed of a soluble material, and wherein the shapeable element is configured to (a) impart a first stiffness to the shunting element during delivery of the shunting element to the target location within the patient and for a first preselected period of time after delivery, and (b) impart a second stiffness less than the first stiffness to the shunting element for a second preselected period of time after the first period of time.

55. The method of claim 51 wherein the shapeable element is composed, at least in part, of hydrogel.

56. The method of claim 51 wherein the shapeable element is composed, at least in part, of polysaccharide.
